(19) United States
(12) Patent Application Publication
(10) Pub. No.: US 2018/0214320 A1
(43) Pub. Date: Aug. 2, 2018

(54) BOND PATTERNS AS A MEANS TO DRIVE HIGH RELIEF AND TOPOGRAPHY IN ELASTIC LAMINATES

(71) Applicant: Kimberly-Clark Worldwide, Inc., Neenah, WI (US)

(72) Inventors: David A. Palzewicz, Canton, GA (US); Eric E. Lennon, Roswell, GA (US)

(21) Appl. No.: 15/749,436

(22) PCT Filed: Sep. 30, 2015
(86) PCT No.: PCT/US2015/053310

§ 371 Date: Jan. 31, 2018

(51) Int. Cl. A61F 13/514; D04H 13/00; B32B 27/32

(52) U.S. Cl. CPC A61F 13/5148; B32B 27/32; D04H 13/00

(57) ABSTRACT

The present invention relates to a nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025 mm/gsm to about 0.050 mm/gsm.

### BACKGROUND OF THE INVENTION

Nonwoven, elastic composites are commonly incorporated into products (e.g., diapers, training pants, garments, etc.) to improve their ability to better fit the contours of the body. For example, the composite may be formed from an elastic film and one or more nonwoven web materials. The nonwoven web material may be joined to the elastic film while the film is in a stretched condition so that the nonwoven web material can gather between the locations where it is bonded to the film when it is relaxed. The resulting elastic composite is stretchable to the extent that the nonwoven web material gathered between the bond locations allows the elastic film to elongate. Unfortunately, elastic films often have unpleasant tactile aesthetic properties, such as feeling rubbery or tacky to the touch, making them unpleasant and uncomfortable against the wearer's skin. In an effort to improve these properties, aperturing the composite has been a viable solution. Although this has helped address concerns for breathability, aesthetic attributes of the composite remain open.

Aesthetics can offer many benefits to the design and overall appearance of a composite. Usually done through high quality printing of graphics, such additions can become costly both to the manufacturer and the end-user. Aesthetics can also offer visual cues to the end-user to assist with the proper functioning and use of a material or garment. Or, they can offer distinguishing patterns that give a more textured or cloth-like appearance which can offer more comfort to the user. A need, therefore, exists to offer an improved elastic composite that possesses the breathability desired, and further, offers the visual cues needed to comfort, distinguish and even direct a user on the proper functioning of an overall product.

### SUMMARY OF THE INVENTION

The present invention relates to a nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025 mm/gsm to about 0.050 mm/gsm.

The present invention also relates to an absorbent article comprising an outer cover, a bodyside liner joined to the outer cover, and an absorbent core positioned between the outer cover and the bodyside liner, wherein the absorbent article includes a nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025 mm/gsm to about 0.050 mm/gsm. Further, the present invention also relates to an absorbent article comprising a nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025 mm/gsm to about 0.050 mm/gsm; wherein the distinct, tactile patterns directionally aids a user in distinguishing the front of the article from the back of the article and/or provides visual cues for proper operation of the absorbent article.

### BRIEF DESCRIPTION OF THE DRAWINGS

A full and enabling disclosure of the present invention is set forth more particularly in the remainder of the specification, including reference to the accompanying figures, in which:

FIG. 1 is a photo image of one embodiment of the present invention which, is also shown in Example 2.

FIG. 2 is a photo image of one embodiment of the present invention which, is also shown in Example 3.

FIG. 3 is a photo image of a comparative nonwoven fabric shown as “rib-knit”.

FIG. 4 is a photo image of a comparative nonwoven fabric shown as “wire weave” which, is also shown in Example 4.

FIG. 5 is a drawing of the bonded and unbonded areas of one embodiment of the present invention.

FIG. 6 is a drawing of the bonded and unbonded areas of one embodiment of the present invention.

FIG. 7 is a photo image of the a 76.2 mm diameter platen at 0.345 kPa (30 psi) loading apparatus used to measure the thickness of a designated area of a nonwoven or composite material in order to determine the caliper to basis weight ratio values of the present invention and comparatives.

### DETAILED DESCRIPTION OF THE INVENTION

### Definitions

As used herein the term “nonwoven web” generally refers to a web having a structure of individual fibers or threads which are interlaid, but not in an identifiable manner as in a knitted fabric. Examples of suitable nonwoven fabrics or webs include, but are not limited to, meltblown webs, spunbond webs, bonded carded webs, airlaid webs, coform webs, hydraulically entangled webs, and so forth.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al. Spunbond fibers are generally not tacky when they are deposited onto a collecting surface. Spunbond fibers may sometimes have diameters less than about 40 microns, and are often between about 5 microns to about 20 microns.

As used herein the terms “extensible” or “extensibility” generally refers to a material that stretches or extends in the direction of an applied force by at least about 25%, in some embodiments about 50%, and in some embodiments, at least about 75% of its relaxed length or width. An extensible material does not necessarily have recovery properties. For example, an elastomeric material is an extensible material having recovery properties. A meltblown web may be extensible, but not have recovery properties, and thus, be an extensible, non-elastic material.

As used herein, the term “elastomeric” and “elastic” and refers to a material that, upon application of a stretching force, is stretchable in at least one direction (such as the CD direction), and which upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, the material contracts or recovers at least 50%, and even more desirably, at least 80% of the stretched length.

As used herein, the terms “machine direction” or “MD” generally refers to the direction in which a material is produced with respect to the length of the material. The term “cross-machine direction” or “CD” refers to the direction perpendicular to the machine direction with or the direction with respect to the width of the material.

As used herein, the terms “necked” and “necked material” generally refer to any material that has been drawn in at least one dimension (e.g., machine direction) to reduce its transverse dimension (e.g., cross-machine direction) so that when the drawing force is removed, the material may be pulled back to its original width. The necked material generally has a higher basis weight per unit area than the un-necked material. When the necked material is pulled back to its original width, it should have about the same basis weight as the un-necked material. This differs from the orientation of a film in which the film is thinned and the basis weight is reduced. The necking method typically involves unwinding a material from a supply roll and passing it through a brake nip roll assembly driven at a given linear speed. A take-up roll or nip, operating at a linear speed higher than the brake nip roll, draws the material and generates the tension needed to elongate and neck the material.

As used herein, the term “thermal point bonding” generally refers to a process performed, for example, by passing a material between a patterned roll (e.g., calender roll) and another roll (e.g., anvil roll), which may or may not be patterned. One or both of the rolls are typically heated.

As used herein, the term “ultrasonic bonding” generally refers to a process performed, for example, by passing a material between a sonic horn and a patterned roll (e.g., anvil roll). For instance, ultrasonic bonding through the use of a stationary horn and a rotating patterned anvil roll is described in U.S. Pat. No. 3,939,033 to Grgach, et al., U.S. Pat. No. 3,844,869 to Rust Jr., and U.S. Pat. No. 4,259,399 to Hill, which are incorporated herein in their entirety by reference thereto for all purposes. Moreover, ultrasonic bonding through the use of a rotary horn with a rotating patterned anvil roll is described in U.S. Pat. No. 5,096,532 to Neuwirth, et al., U.S. Pat. No. 5,110,403 to Ehlert, and U.S. Pat. No. 5,817,199 to Brennecke, et al., which are incorporated herein in their entirety by reference thereto for all purposes. Of course, any other ultrasonic bonding technique may also be used in the present invention.

It is to be noted that the terms “bonding” and “lamination” or “laminating” may be used interchangeably to refer to the process by which the film is affixed to the nonwoven web.

As used herein, the term “distinct, tactile pattern” refers to the pattern created by the unbonded areas having some three-dimensionality or a z-directional component that is noticeable by vision or even by touch to a user of the product. The depth of the z-directional component of the patterns may be the same or somewhat similar in magnitude to optimize the noticeability to a user of the product. However, it is not uncommon that a few of the patterns can differ in depth, particularly if combinations of shapes are used in the overall nonwoven composite. The distinct, tactile patterns consist essentially of shaped elements created by the unbonded areas such as flowers, squares, circles, stars, zig-zags, arrows, cartoon characters, faces, balloons, animals, nature, waves, swirls, rectangles, ovals, triangles, diamonds, polygons, and abstract shapes, caricatures, and the like.

The use of any trademarks herein has been noted with CAPITALIZATION of the word wherever it appears to acknowledge and respect the proprietary nature held by the owners of the mark. If necessary, the word is followed by the generic terminology only wherever it appears for the first time herein.

### DETAILED DESCRIPTION

While the specification concludes with the claims particularly pointing out and distinctly claiming the invention, it is believed that the present invention will be better understood from the following description.

Generally speaking, the present invention relates to a nonwoven composite comprising an elastic film wherein the film is strategically positioned to remain unbonded to the nonwoven web enable a distinct, tactile three-dimensional surface.

The nonwoven composite comprises an elastic film laminated to one or more nonwoven web materials wherein due to the angles and spacing of the bonding, a voided space is left from the unbonded web area to create a patterned, tactile feature resulting in a nonwoven composite with aesthetic and functional visual cues. The composite is formed by passing the film through a nip specifically patterned with varying bonding pins specifically oriented and arranged to bond the film to the nonwoven web material(s). Apertures may or may not be present in the elastic film. If apertures are desired to be form, the apertures are of a size sufficient to provide a desired level of texture, softness, hand feel, and/or aesthetic appeal to the composite without having a significant adverse effect on its elastic properties. Aperture and bond formation are accomplished in the present invention by selectively controlling certain parameters of the lamination process, such as film content, bonding pattern, degree of film tension, bonding conditions, etc. In this regard, various embodiments of the present invention will now be described in more detail.

### I. Elastic Film

The elastic film of the present invention is formed from one or more elastomeric polymers that are melt-processable, i.e. thermoplastic. Any of a variety of thermoplastic elastomeric polymers may generally be employed in the present invention, such as elastomeric polyesters, elastomeric polyurethanes, elastomeric polyamides, elastomeric copolymers, elastomeric polyolefins, and so forth. In one particular embodiment, elastomeric semi-crystalline polyolefins are employed due to their unique combination of mechanical and elastomeric properties. That is, the mechanical properties of such semi-crystalline polyolefins allows for the formation of films that readily aperture during thermal bonding, but yet retain their elasticity.

Semi-crystalline polyolefins have or are capable of exhibiting a substantially regular structure. For example, semi-crystalline polyolefins may be substantially amorphous in their undeformed state, but form crystalline domains upon stretching. Exemplary semi-crystalline polyolefins include polyethylene, polypropylene, blends and copolymers thereof. Particularly suitable polyethylene copolymers are those that are “linear” or “substantially linear.” The term “substantially linear” means that, in addition to the short chain branches attributable to comonomer incorporation, the ethylene polymer also contains long chain branches in that the polymer backbone. “Long chain branching” refers to a chain length of at least 6 carbons. Each long chain branch may have the same comonomer distribution as the polymer backbone and be as long as the polymer backbone to which it is attached. In contrast to the term “substantially linear”, the term “linear” means that the polymer lacks measurable or demonstrable long chain branches. That is, the polymer is substituted with an average of less than 0.01 long chain branch per 1000 carbons.

Although not necessarily required, linear polyethylene “plastomers” are particularly desirable in that the content of α-olefin short chain branching content is such that the ethylene copolymer exhibits both plastic and elastomeric characteristics—i.e., a “plastomer.” Because polymerization with α-olefin comonomers decreases crystallinity and density, the resulting plastomer normally has a density lower than that of polyethylene thermoplastic polymers (e.g., LLDPE), but approaching and/or overlapping that of an elastomer. Despite having a density similar to elastomers, plastomers generally exhibit a higher degree of crystallinity, are relatively non-tacky, and may be formed into pellets that are non-adhesive and relatively free flowing. Preferred plastomers for use in the present invention are ethylene-based copolymer plastomers available under the designation EXACT from ExxonMobil Chemical Company of Houston, Tex. Other suitable polyethylene plastomers are available under the designation INFUSE, ENGAGE and AFFINITY from Dow Chemical Company of Midland, Mich. Still other suitable ethylene polymers are available from The Dow Chemical Company under the designations DOWLEX (LLDPE) and ATTANE (ULDPE). Other suitable ethylene polymers are described in U.S. Pat. No. 4,937,299 to Ewen et al.; U.S. Pat. No. 5,218,071 to Tsutsui et al.; U.S. Pat. No. 5,272,236 to Lai, et al.; and U.S. Pat. No. 5,278,272 to Lai, et al. Of course, the present invention is by no means limited to the use of ethylene polymers. For instance, propylene polymers may also be suitable for use as a semi-crystalline polyolefin. Suitable plastomeric propylene polymers may include, for instance, copolymers or terpolymers of propylene include copolymers of propylene with an α-olefin (e.g., C₃-C₂₀), such as ethylene, 1-butene, 2-butene, the various pentene isomers, 1-hexene, 1-octene, 1-nonene, 1-decene, 1-unidecene, 1-dodecene, 4-methyl-1-pentene, 4-methyl-1-hexene, 5-methyl-1-hexene, vinylcyclohexene, styrene, etc. The comonomer content of the propylene polymer may be about 35 wt. % or less, in some embodiments from about 1 wt. % to about 20 wt. %, and in some embodiments, from about 2 wt. % to about 10 wt. %. Preferably, the density of the polypropylene (e.g., propylene/α-olefin copolymer) may be 0.91 grams per cubic centimeter (g/cm³) or less, in some embodiments, from 0.85 to 0.88 g/cm³, and in some embodiments, from 0.85 g/cm³ to 0.87 g/cm³. Suitable propylene polymers are commercially available under the designations VISTAMAXX from ExxonMobil Chemical Co. of Houston, Tex.; FINA™ (e.g., 8573) from Atofina Chemicals of Feluy, Belgium; TAFMER available from Mitsui Petrochemical Industries; and VERSIFY available from Dow Chemical Co. of Midland, Michigan. Other examples of suitable propylene polymers are described in U.S. Pat. No. 6,500,563 to Datta, et al.; U.S. Pat. No. 5,539,056 to Yang, et al.; and U.S. Pat. No. 5,596,052 to Resconi, et al.

Of course, other thermoplastic polymers may also be used to form the elastic film, either alone or in conjunction with the semi-crystalline polyolefins. For instance, a substantially amorphous block copolymer may be employed that has at least two blocks of a monoalkenyl arene polymer separated by at least one block of a saturated conjugated diene polymer. The monoalkenyl arene blocks may include styrene and its analogues and homologues, such as o-methyl styrene; p-methyl styrene; p-tert-butyl styrene; 1,3 dimethyl styrene p-methyl styrene, etc. . . . , as well as other monoalkenyl polycyclic aromatic compounds, such as vinyl naphthalene; vinyl anthrycene; and so forth. Preferred monoalkenyl arenes are styrene and p-methyl styrene. The conjugated diene blocks may include homopolymers of conjugated diene monomers, copolymers of two or more conjugated dienes, and copolymers of one or more of the dienes with another monomer in which the blocks are predominantly conjugated diene units. Preferably, the conjugated dienes contain from 4 to 8 carbon atoms, such as 1,3 butadiene (butadiene); 2-methyl-1,3 butadiene; isoprene; 2,3 dimethyl-1,3 butadiene; 1,3 pentadiene (piperylene); 1,3 hexadiene; and so forth.

Particularly suitable thermoplastic elastomeric copolymers are available from Kraton Polymers LLC of Houston, Tex. under the trade name KRATON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Patent No. 5,332,613 to Taylor, et al.

The amount of elastomeric polymer(s) employed in the film may vary, but is typically about 30 wt. % or more of the film, in some embodiments about 50 wt. % or more, and in some embodiments, about 80 wt. % or more of the of the film. In one embodiment, for example, the semi-crystalline polyolefin(s) constitute about 70 wt. % or more of the film, in some embodiments about 80 wt. % or more of the film, and in some embodiments, about 90 wt. % or more of the film. In other embodiments, blends of semi-crystalline polyolefin(s) and elastomeric block copolymer(s) may be employed. In such embodiments, the block copolymer(s) may constitute from about 5 wt. % to about 50 wt. %, in some embodiments from about 10 wt. % to about 40 wt. %, and in some embodiments, from about 15 wt. % to about 35 wt. % of the blend. Likewise, the semi-crystalline polyolefin(s) may constitute from about 50 wt. % to about 95 wt. %, in some embodiments from about 60 wt. % to about 90 wt. %, and in some embodiments, from about 65 wt. % to about 85 wt. % of the blend. It should of course be understood that other elastomeric and/or non-elastomeric polymers may also be employed in the film.

Besides polymers, the elastic film of the present invention may also contain other components as is known in the art. In one embodiment, for example, the elastic film contains a filler. Fillers are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6,111,163 to McCormack, et al.; U.S. Pat. No. 5,932,497 to Morman, et al.; U.S. Pat. No. 6,461,457 to Taylor, et al. Examples of suitable fillers include, but are not limited to, calcium carbonate, various kinds of clay, silica, alumina, barium carbonate, sodium carbonate, magnesium carbonate, talc, barium sulfate, magnesium sulfate, aluminum sulfate, titanium dioxide, zeolites, cellulose-type powders, kaolin, mica, carbon, calcium oxide, magnesium oxide, aluminum hydroxide, pulp powder, wood powder, cellulose derivatives, chitin and chitin derivatives. Other additives may also be incorporated into the film, such as melt stabilizers, processing stabilizers, heat stabilizers, light stabilizers, antioxidants, heat aging stabilizers, whitening agents, antiblocking agents, bonding agents, tackifiers, viscosity modifiers, etc.

The elastic film of the present invention may be mono- or multi-layered. Multilayer films may be prepared by co-extrusion of the layers, extrusion coating, or by any conventional layering process. Such multilayer films normally contain at least one base layer and at least one skin layer, but may contain any number of layers desired. For example, the multilayer film may be formed from a base layer and one or more skin layers, wherein the base layer is formed from a semi-crystalline polyolefin. In such embodiments, the skin layer(s) may be formed from any film-forming polymer. If desired, the skin layer(s) may contain a softer, lower melting polymer or polymer blend that renders the layer(s) more suitable as heat seal bonding layers for thermally bonding the film to a nonwoven web. For example, the skin layer(s) may be formed from an olefin polymer or blends thereof, such as described above. Additional film-forming polymers that may be suitable for use with the present invention, alone or in combination with other polymers, include ethylene vinyl acetate, ethylene ethyl acrylate, ethylene acrylic acid, ethylene methyl acrylate, ethylene normal butyl acrylate, nylon, ethylene vinyl alcohol, polystyrene, polyurethane, and so forth.

The properties of the resulting film may generally vary as desired. For instance, prior to stretching, the film typically has a basis weight of about 100 grams per square meter or less, and in some embodiments, from about 50 to about 75 grams per square meter. Upon stretching, the film typically has a basis weight of about 60 grams per square meter or less, and in some embodiments, from about 15 to about 35 grams per square meter. The stretched film may also have a total thickness of from about 1 to about 100 micrometers, in some embodiments, from about 10 to about 80 micrometers, and in some embodiments, from about 20 to about 60 micrometers.

### II. Nonwoven Web Material

The polymers used to form the nonwoven web material typically have a softening temperature that is higher than the temperature imparted during bonding. In this manner, the polymers do not substantially soften during bonding to such an extent that the fibers of the nonwoven web material become completely melt flowable. For instance, polymers may be employed that have a Vicat softening temperature (ASTM D-1525) of from about 100° C. to about 300° C., in some embodiments from about 120° C. to about 250° C., and in some embodiments, from about 130° C. to about 200° C. Exemplary high-softening point polymers for use in forming nonwoven web materials may include, for instance, polyolefins, e.g., polyethylene, polypropylene, polybutylene, etc.; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; and so forth. If desired, biodegradable polymers, such as those described above, may also be employed. Synthetic or natural cellulosic polymers may also be used, including but not limited to, cellulosic esters; cellulosic ethers; cellulosic nitrates; cellulosic acetates; cellulosic acetate butyrates; ethyl cellulose; regenerated celluloses, such as viscose, rayon, and so forth. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven web material. Monocomponent fibers are generally formed from a polymer or blend of polymers extruded from a single extruder. Multicomponent fibers are generally formed from two or more polymers (e.g., bicomponent fibers) extruded from separate extruders. The polymers may be arranged in substantially constantly positioned distinct zones across the cross-section of the fibers. The components may be arranged in any desired configuration, such as sheath-core, side-by-side, pie, island-in-the-sea, three island, bull's eye, or various other arrangements known in the art. and so forth. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference thereto for all purposes. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference thereto for all purposes.

Although any combination of polymers may be used, the polymers of the multicomponent fibers are typically made from thermoplastic materials with different glass transition or melting temperatures where a first component (e.g., sheath) melts at a temperature lower than a second component (e.g., core). Softening or melting of the first polymer component of the multicomponent fiber allows the multicomponent fibers to form a tacky skeletal structure, which upon cooling, stabilizes the fibrous structure.

Fibers of any desired length may be employed, such as staple fibers, continuous fibers, etc. In one particular embodiment, for example, staple fibers may be used that have a fiber length in the range of from about 1 to about 150 millimeters, in some embodiments from about 5 to about 50 millimeters, in some embodiments from about 10 to about 40 millimeters, and in some embodiments, from about 10 to about 25 millimeters. Although not required, carding techniques may be employed to form fibrous layers with staple fibers as is well known in the art. For example, fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be bonded using known techniques to form a bonded carded nonwoven web.

If desired, the nonwoven web material used to form the nonwoven composite may have a multi-layer structure. Suitable multi-layered materials may include, for instance, spunbond/meltblown/spunbond (SMS) laminates and spunbond/meltblown (SM) laminates. Various examples of suitable SMS laminates are described in U.S. Pat. No. 4,041,203 to Brock et al.; U.S. Pat. No. 5,213,881 to Timmons, et al.; U.S. Pat. No. 5,464,688 to Timmons, et al.; U.S. Pat. No. 4,374,888 to Bornslaeger; 5,169,706 to Collier, et al.; and U.S. Pat. No. 4,766,029 to Brock et al. In addition, commercially available SMS laminates may be obtained from Kimberly-Clark Corporation under the designations Spunguard® and Evolution®. Another example of a multi-layered structure is a spunbond web produced on a multiple spin bank machine in which a spin bank deposits fibers over a layer of fibers deposited from a previous spin bank. Such an individual spunbond nonwoven web may also be thought of as a multi-layered structure. In this situation, the various layers of deposited fibers in the nonwoven web may be the same, or they may be different in basis weight and/or in terms of the composition, type, size, level of crimp, and/or shape of the fibers produced. As another example, a single nonwoven web may be provided as two or more individually produced layers of a spunbond web, a carded web, etc., which have been bonded together to form the nonwoven web. These individually produced layers may differ in terms of production method, basis weight, composition, and fibers as discussed above. The nonwoven web material of the present invention may comprise spunbond fibers, meltblown fibers, staple fibers, or combinations thereof. Overall, the basis weight of the nonwoven web material should be at least about 20 gsm or at least about 25 gsm. Lower basis weight materials can be used, however, after a higher basis weight creates a better visual cue and higher loft. The overall composite will also vary from at least about 80 gsm or at least about 100 gsm or at least about 115 gsm or at least about 125 gsm.

A nonwoven web material may also contain an additional fibrous component such that it is considered a composite. For example, a nonwoven web may be entangled with another fibrous component using any of a variety of entanglement techniques known in the art (e.g., hydraulic, air, mechanical, etc.). In one embodiment, the nonwoven web is integrally entangled with cellulosic fibers using hydraulic entanglement. A typical hydraulic entangling process utilizes high pressure jet streams of water to entangle fibers to form a highly entangled consolidated fibrous structure, e.g., a nonwoven web. Hydraulically entangled nonwoven webs of staple length and continuous fibers are disclosed, for example, in U.S. Pat. No. 3,494,821 to Evans and U.S. Pat. No. 4,144,370 to Boulton. Hydraulically entangled composite nonwoven webs of a continuous fiber nonwoven web and a pulp layer are disclosed, for example, in U.S. Pat. No. 5,284,703 to Everhart, et al. and U.S. Pat. No. 6,315,864 to Anderson, et al.

Although not required, the nonwoven web material may be necked in one or more directions prior to bonding to the film of the present invention. Suitable techniques necking techniques are described in U.S. Pat. Nos. 5,336,545, 5,226,992, 4,981,747 and 4,965,122 to Morman, as well as U.S. Patent Application Publication No. 2004/0121687 to Morman, et al. Alternatively, the nonwoven web may remain relatively inextensible in at least one direction prior to bonding to the film. In such embodiments, the nonwoven web may be optionally stretched in one or more directions subsequent to bonding to the film.

### III. Lamination Technique

Lamination is generally accomplished in the present invention via a bonding technique (e.g., thermal point bonding, ultrasonic bonding, etc.) in which the materials are supplied to a nip defined by at least one patterned roll. Thermal point bonding, for instance, typically employs a nip formed between two rolls, at least one of which is patterned. Ultrasonic bonding, on the other hand, typically employs a nip formed between a sonic horn and a patterned roll. Regardless of the technique chosen, the patterned roll contains a plurality of raised bonding elements to concurrently bond the film to the nonwoven web material(s). The size of the bonding elements may be specifically tailored to facilitate and enhance bonding between the film and the nonwoven material(s). For example, the bonding elements are typically selected to have a relatively large length dimension. The length dimension of the bonding elements may be from about 300 to about 5000 micrometers, in some embodiments from about 500 to about 4000 micrometers, and in some embodiments, from about 1000 to about 2000 micrometers. The width dimension of the bonding elements may likewise range from about 20 to about 500 micrometers, in some embodiments from about 40 to about 200 micrometers, and in some embodiments, from about 50 to about 150 micrometers. In addition, the “element aspect ratio” (the ratio of the length of an element to its width) may range from about 2 to about 100, in some embodiments from about 4 to about 50, and in some embodiments, from about 5 to about 20.

Should apertures be employed, the bonding elements may also be tailored to form the apertures in the film so that apertures and bonds between the film and the nonwoven web material can occur concurrently. In one embodiment, for example, the longitudinal axis (longest dimension along a center line of the element) of one or more of the bonding elements is skewed relative to the machine direction (“MD”) of the elastic film. For example, one or more of the bonding elements may be oriented from about 30° to about 150°, in some embodiments from about 45° to about 135°, and in some embodiments, from about 60° to about 120° relative to the machine direction of the film. In this manner, the bonding elements will present a relatively large surface to the film in a direction substantially perpendicular to that which the film moves. This increases the area over which shear stress is imparted to the film and, in turn, facilitates aperture formation.

To achieve concurrent aperture and bond formation without substantially softening the polymer(s) of the nonwoven web material, the bonding temperature and pressure may be selectively controlled. For example, one or more rolls may be heated to a surface temperature of from about 50° C. to about 160° C., in some embodiments from about 60° C. to about 140° C., and in some embodiments, from about 70° C. to about 120° C. Likewise, the pressure exerted by rolls (“nip pressure”) during thermal bonding may range from about 75 to about 600 pounds per linear inch, in some embodiments from about 100 to about 400 pounds per linear inch, and in some embodiments, from about 120 to about 200 pounds per linear inch. Of course, the residence time of the materials may influence the particular bonding parameters employed.

As stated, another factor that influences concurrent aperture and bond formation is the degree of tension in the film during lamination. An increase in film tension, for example, typically correlates to an increase in aperture size. Of course, a film tension that is too high may adversely affect the integrity of the film. Thus, in most embodiments of the present invention, a stretch ratio of about 1.5 or more, in some embodiments from about 2.5 to about 7.0, and in some embodiments, from about 3.0 to about 5.5, is employed to achieve the desired degree of tension in the film during lamination. The stretch ratio may be determined by dividing the final length of the film by its original length. The stretch ratio may also be approximately the same as the draw ratio, which may be determined by dividing the linear speed of the film during lamination (e.g., speed of the nip rolls) by the linear speed at which the film is formed (e.g., speed of casting rolls or blown nip rolls).

The selection of an appropriate bonding temperature (e.g., the temperature of a heated roll) will help melt and/soften the low-softening point elastomeric polymer(s) of the film at regions adjacent to the bonding elements. The softened elastomeric polymer(s) may then flow and become displaced during bonding, such as by pressure exerted by the bonding elements. The displaced portions of the film surrounding the apertures can also fuse to the nonwoven web material(s), thereby forming an integral nonwoven composite. Furthermore, because the elastomeric polymer(s) may physically entrap or adhere to the fibers at the bond sites, adequate bond formation may be achieved without requiring substantial softening of the polymer(s) used to form the nonwoven web material. Thus, the nonwoven web material remains substantially unbonded to the film or other materials to create the visually distinct, tactile pattern.

The film may be “pre-stretched” (prior to lamination) by rolls rotating at different speeds of rotation so that the sheet is stretched to the desired stretch ratio in the machine direction. This uniaxially stretched film may also be oriented in the cross-machine direction to form a “biaxially stretched” film. The orientation temperature profile during the “pre-stretching” operation is generally below the melting point of one or more polymers in the film, but high enough to enable the composition to be drawn or stretched. For example, the film may be stretched at a temperature from about 15° C. to about 50° C., in some embodiments from about 25° C. to about 40° C., and in some embodiments, from about 30° C. to about 40° C. When “pre-stretched” in the manner described above, the degree of stretch during lamination may be increased, maintained, or slightly reduced (retracted) to desired degree of tension.

A method for forming a composite from an elastic film and a nonwoven web material may be to take the raw materials of the film (e.g., elastomeric polymer) and dry mix them together (i.e., without a solvent) then add to a hopper of an extrusion apparatus. The raw materials may alternatively be blended with a solvent. In the hopper, the materials are dispersively mixed in the melt and compounded using any known technique, such as batch and/or continuous compounding techniques that employ, for example, a Banbury mixer, Farrel continuous mixer, single screw extruder, twin screw extruder, etc.

Any known technique may be used to form a film from the compounded material, including blowing, casting, flat die extruding, etc. For example, the film may be formed by a blown process in which a gas (e.g., air) is used to expand a bubble of the extruded polymer blend through an annular die. The bubble is then collapsed and collected in flat film form. Processes for producing blown films are described, for instance, in U.S. Pat. No. 3,354,506 to Raley; U.S. Pat. No. 3,650,649 to Schippers; and U.S. Pat. No. 3,801,429 to Schrenk et al., as well as U.S. Patent Application Publication Nos. 2005/0245162 to McCormack, et al. and 2003/0068951 to Boggs, et al. For example, the compounded material may be supplied to an extrusion apparatus and then blown into nip rolls 42 to form a single-layered precursor elastic film. The rolls may be kept at a temperature sufficient to solidify and quench the precursor elastic film as it is formed, such as from about 20° C. to about 60° C. Typically, the resulting precursor elastic film is generally unapertured, although it may of course possess small cuts or tears as a result of processing. The use of an unapertured film can provide a variety of benefits, including the avoidance of registration steps needed to align the apertures with bond sites during lamination.

One method for forming a uniaxially stretched film is stretch and thin the film in the machine direction by passing it through a film-orientation unit or machine direction orienter (“MDO”), such as commercially available from Marshall and Willams, Co. of Providence, R.I. The film may be stretched in either single or multiple discrete stretching operations. The film may also be stretched in other directions. For example, the film may be clamped at its lateral edges by chain clips and conveyed into a tenter oven. In the tenter oven, the film may be drawn in the cross-machine direction to the desired stretch ratio by chain clips diverged in their forward travel.

A nonwoven web material is also employed for laminating to the elastic film. For example, the nonwoven web material may simply be unwound from a supply roll. Alternatively, a nonwoven web material may be formed in-line, such as by spunbond extruders. The extruders may deposit fibers onto a forming wire, which is part of a continuous belt arrangement that circulates around a series of rolls. If desired, a vacuum may be utilized to maintain the fibers on the forming wire. The spunbond fibers form a mat that may optionally be compressed via compaction rolls. Although not necessarily required, a second material originating from a supply roll may also be laminated to the elastic film. The second material may be a second nonwoven web material, film, etc.

Regardless, thermal bonding techniques are employed to laminate the material(s) to the elastic film. For instance, the materials may be directed to a nip defined between rolls for laminating to the elastic film. One or both of the rolls may contain a plurality of raised bonding elements and/or may be heated. Upon lamination, the elastic film is melt fused to the nonwoven web materials at a plurality of discrete bond sites. That is, the elastomeric polymer(s) of the film are softened and/or melted so that they may physically entrap fibers of the nonwoven web materials. Of course, the elastic film may possess a certain tack so that it also adheres to the fibers upon lamination. The bond sites may be located proximate (adjacent or near to) a perimeter defined by corresponding apertures, which are formed by displacement of the film. The particular location of the bond sites adjacent to or near the apertures may enhance the integrity of the resulting composite by strengthening the area surrounding the apertures. Because thermal bonding occurs at a temperature that is insufficient to substantially soften the polymer(s) of the nonwoven web materials, as described above, they are not substantially melt fused to each other. In this manner, the composite may better retain the physical properties (e.g., liquid permeability, softness, bulk, and hand feel) of the individual nonwoven web materials.

The resulting composite may then be wound and stored on a take-up roll. Optionally, the composite may be kept under tension, such as by using the same linear velocity for the roll as the speed of one or more of the stretching rolls. More preferably, however, the composite is allowed to slightly retract prior to winding on to the take-up roll. This may be achieved by using a slower linear velocity for the roll. Because the elastic film is tensioned prior to lamination, it will retract toward its original machine direction length and become shorter in the machine direction, thereby buckling or forming gathers in the composite. The resulting elastic composite thus becomes extensible in the machine direction to the extent that the gathers or buckles in the web may be pulled back out flat and allow the elastic film to elongate.

Various additional potential processing and/or finishing steps known in the art, such as slitting, treating, printing graphics, etc., may be performed without departing from the spirit and scope of the invention. For instance, the composite may optionally be mechanically stretched in the cross-machine and/or machine directions to enhance extensibility. In one embodiment, the composite may be coursed through two or more rolls that have grooves in the CD and/or MD directions. Such grooved satellite/anvil roll arrangements are described in U.S. Patent Application Publication Nos. 2004/0110442 to Rhim, et al. and 2006/0151914 to Gerndt, et al. For instance, the laminate may be coursed through two or more rolls that have grooves in the CD and/or MD directions. The grooved rolls may be constructed of steel or other hard material (such as a hard rubber). If desired, heat may be applied to the composite just prior to or during the application of incremental stretch to cause it to relax somewhat and ease extension. Heat may be applied by any suitable method known in the art, such as heated air, infrared heaters, heated nipped rolls, or partial wrapping of the laminate around one or more heated rolls or steam canisters, etc. Heat may also be applied to the grooved rolls themselves. It should also be understood that other grooved roll arrangement are equally suitable, such as two grooved rolls positioned immediately adjacent to one another.

Besides the above-described grooved rolls, other techniques may also be used to mechanically stretch the composite in one or more directions. For example, the composite may be passed through a tenter frame that stretches the composite. Such tenter frames are well known in the art and described, for instance, in U.S. Patent Application Publication No. 2004/0121687 to Morman, et al. The composite may also be necked. Suitable techniques necking techniques are described in U.S. Pat. Nos. 5,336,545, 5,226,992, 4,981,747 and 4,965,122 to Morman, as well as U.S. Patent Application Publication No. 2004/0121687 to Morman, et al., all of which are incorporated herein in their entirety by reference thereto for all purposes.

### IV. Unbonded Patterning

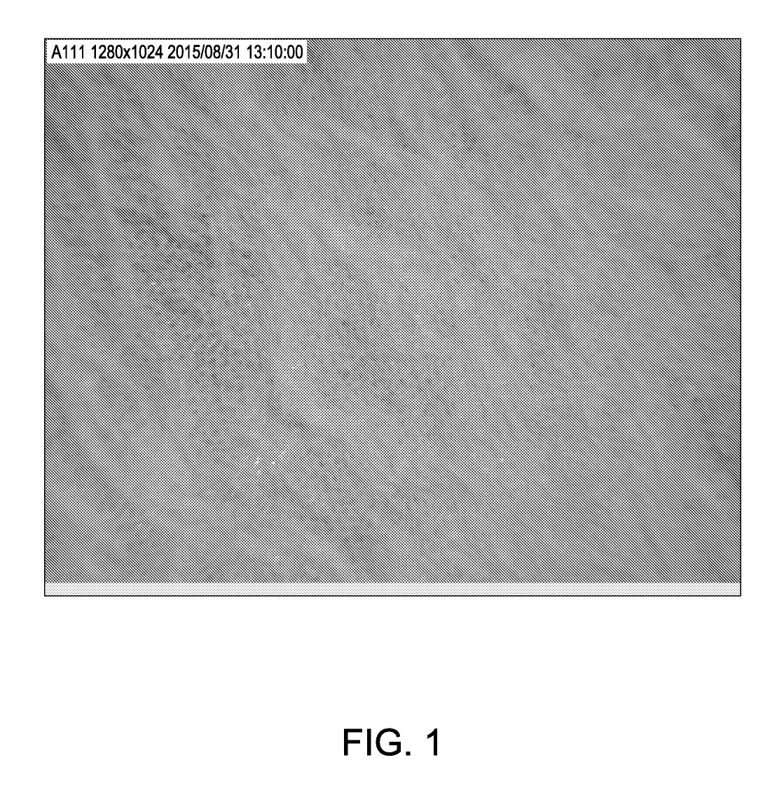

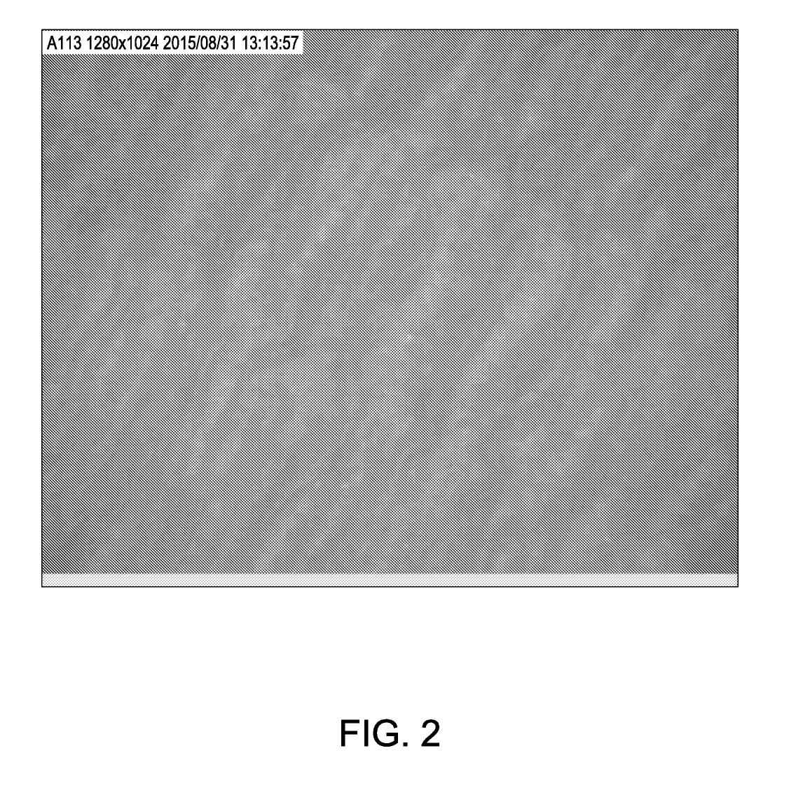

The present invention relates to a nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025 mm/gsm to about 0.050 mm/gsm. The advantages of the present invention can be mostly appreciated by the unbonded areas of the nonwoven web and the film resulting in a distinct, textile pattern in the overall nonwoven composite as shown in FIGS. 1 and 2. Such distinct, textile patterns can not only provide a visual cue to the end user but can also provide functional and directional aids to the user identifying, for example, where to grasp an absorbent article to place it on a wearer or, for example, distinguishing the front side of the article from the back. Thus, an absorbent article comprising a front panel, two side panels, and a back panel may comprise the nonwoven composite of the present invention wherein the distinct, tactile pattern directionally aids the user of the article to distinguish the front panel from the back panel for proper use of the absorbent article. Various shapes that can be felt by touch of the nonwoven composite of the present invention provide necessary identifiers that offer the functional and aesthetically pleasing advantages of the present invention.

The bonding pins utilized in the present invention may be fewer and smaller than pins used as bonding elements in other applications. For example, the pins may be from about 0.020 inches (0.51 mm), from about 0.030 inches (0.762 mm) or from about 0.040 inches (1.02 mm) to about 0.050 inches (1.27 mm). The area of the pins should be from about 0.0004 in² (0.01 mm²) to about 0.005 in² (0.13 mm²), in some embodiments from about 0.004 in² (0.01 mm²) to about 0.0025 in² (0.06 mm²), in some embodiments from about 0.007 in² (0.18 mm²) to about 0.0025 in² (0.06 mm²), and in some embodiments from about 0.004 in² (0.01 mm²) to about 0.007 in² (0.18 mm²). The pin density may be from about 200 pins/in² (5080 pins/mm²) to about 400 pins/in² (10,160 pins/mm²). The pin shape can vary including but not limited to circles, oval, rectangles, stars, squares, and the like.

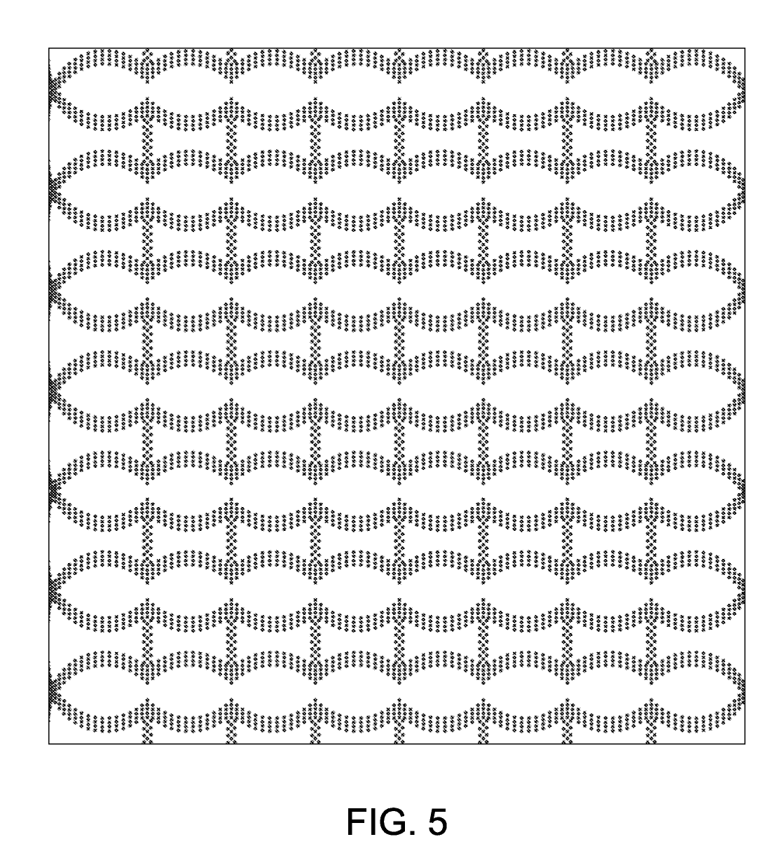

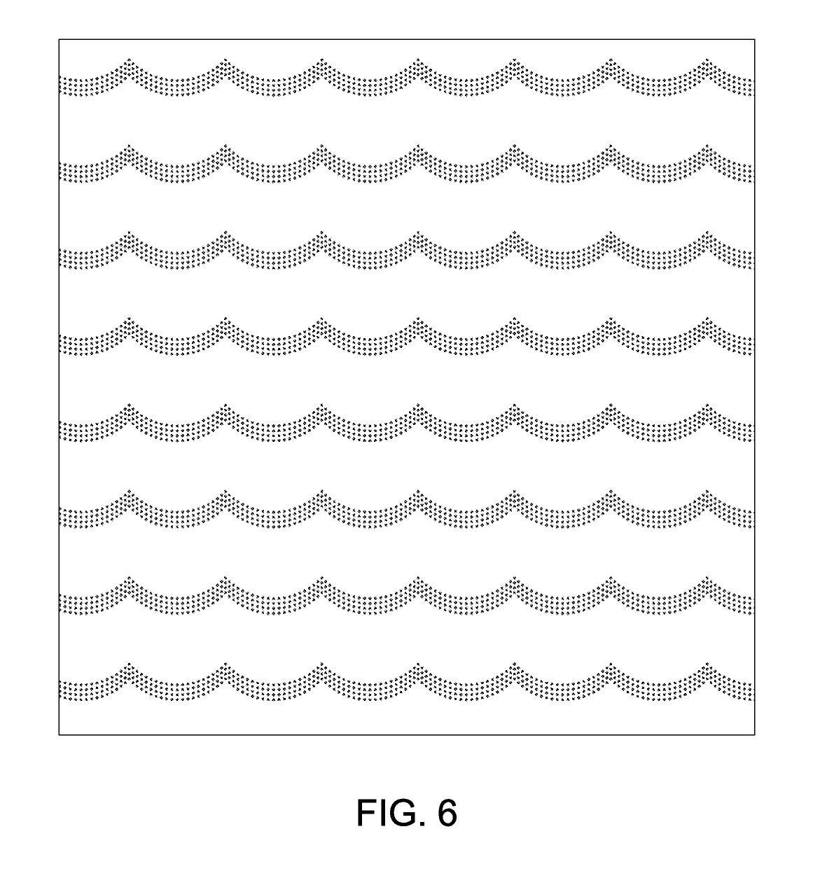

The bonding points of the present invention stem from the strategic placement of the pins to cause an overall response in the unbonded film and unbonded nonwoven web such that a pattern is created. The unbonded portion of the film creates the distinct, tactile pattern within the composite as a result of the film not being bonded to the web. It is to be noted that if apertures are desired, they may be created by the orientation of the pins relative to the machine direction. The unbonded region that gives the distinct, tactile pattern may be from about 20% or from about 35% or from about 50% or from about 60% to about 60% or to about 75% or to about 85% of the overall nonwoven composite. Additionally, the unbonded areas are found in a continuous and particular area of spacing repeated throughout the composite. In other words, the unbonded areas are not minimal. Neither are the unbonded areas a mere adjacent happenstance of the bonding of the film and nonwoven material. They are clearly purposeful and distinguishable patterned regions of the composite that are unbonded. It is to be noted that unlike prior applications where emphasis was placed on the placement, position and percent of area bonded, the present application has strategic positioning of the pins as a focus of the unbonded areas as seen in FIGS. 5 and 6. While inherently, applications with a certain parameter of bonding points would also have unbonded areas, the present invention is distinguishable in that the unbonded areas are specifically defined shapes and patterns to function as distinct and tactile visual cues. In other words, the unbonded areas are not merely spaces that are found in between the bonded areas. As stated earlier, the bonding pins will be fewer and smaller than normally found in prior applications. As such, there has been no appreciation or teaching on how to optimize the unbonded areas such that the high loft topography of the unbonded areas can be seen or felt for a visual appeal or functionally directional benefit. The present invention enhances the overall nonwoven composite without the need for additional graphics that would normally provide functional and visual cues. Thus, the present invention has discovered a means for delivering a functional and aesthetically-pleasing capability unfounded in prior nonwoven and film applications.

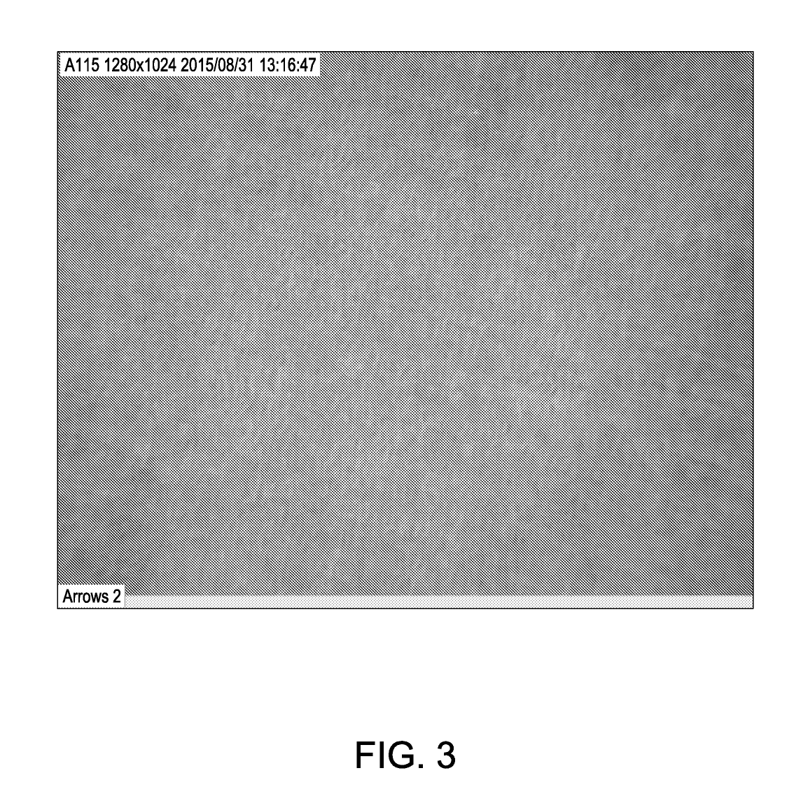

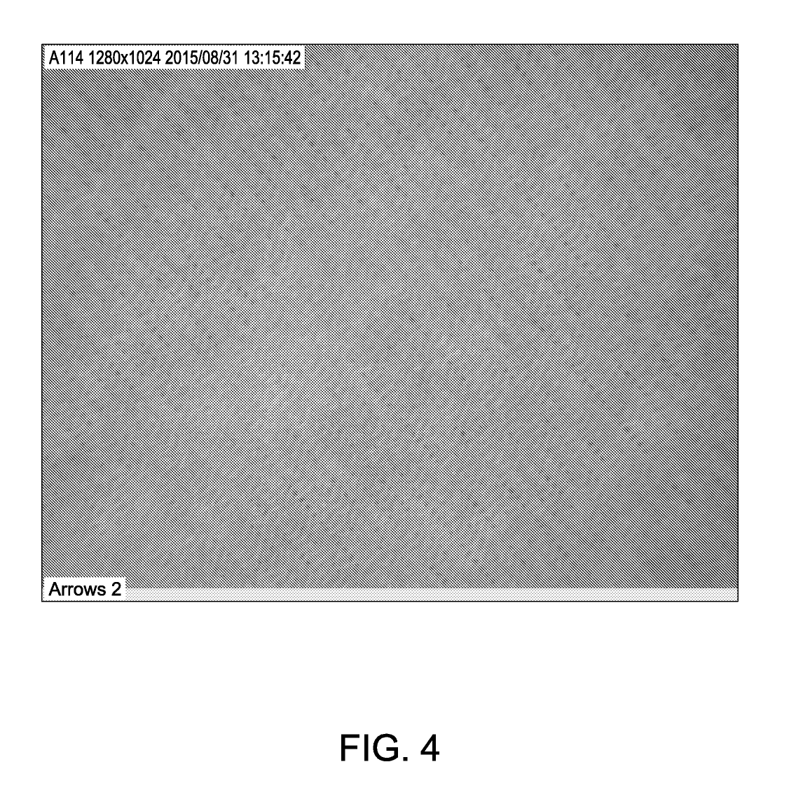

The bonding patterns of the present invention show tremendous improvements over prior patterns such as the “S-weave” pattern as described in U.S. Pat. No. 5,964,742 to McCormack, et al., the “rib-knit” pattern as shown in FIG. 3 and described in U.S. Pat. No. 5,620,779 to Levy, et al., the “wire weave” pattern as shown in FIG. 4, or those described in U.S. Pat. No. 3,855,046 to Hansen et al.; U.S. Pat. No. 5,962,112 to Haynes et al.; U.S. Pat. No. 6,093,665 to Sayovitz et al.; U.S. Pat. No. D375,844 to Edwards, et al.; U.S. Pat. No. D428,267 to Romano et al.; and U.S. Pat. No. D390,708 to Brown. In these applications, the unbonded areas were not distinguishably identifiable or driven by the distinct, tactile patterns stated herein. To indicate the key improvements of the present invention, the caliper of the fabric may be considered using the test herein. The caliper values can be compared, particularly if the nonwoven materials possess the same basis weight. In such case, the caliper of the present invention presents a larger caliper. Having a higher caliper is significant in providing the advantages of the present invention so that a user can employ the tactile properties for functional benefits. Comparatively, a rib knit pattern has a caliper of about 1.3 mm to about 1.4 mm and a wire weave pattern has a caliper of about 1.0 mm to about 1.1 mm. The present invention may provide a caliper measurement as high as about 5.5 mm. In fact, the present invention may provide calipers from about 2.0 mm, from about 2.1 mm, from about 2.5 mm, from about 2.7 mm, to about 3.1 mm, to about 4.8 mm, to about 5.2 mm or to about 5.5 mm.

It is more likely that nonwoven materials may vary in basis weights and thus, a more realistic and discernible approach in accounting for the caliper of the fabric is to look at the ratio of caliper to basis weight. The caliper ratio can be determined by taking the caliper of the fabric and dividing by the basis weight to give a value of mm/gsm. For example, a rib knit pattern has a caliper to basis weight ratio of from about 0.015 mm/gsm to about 0.016 mm/gsm and a wire weave pattern has a caliper to basis weight ratio of from about 0.012 mm/gsm to about 0.013 mm/gsm. Advantageously, the present invention may provide a caliper measurement as high as about 0.050 mm/gsm. In fact, the present invention may provide a caliper to basis weight ratio of from about 0.025 mm/gsm, from about 0.030 mm/gsm, from about 0.032 mm/gsm, from about 0.045 mm/gsm, from about 0.047 mm/gsm to about 0.025 mm/gsm, to about 0.036 mm/gsm, to about 0.045 mm/gsm, to about 0.050 mm/gsm.

Because it is appreciated that the nonwoven composite of the present invention will have a combination of calipers due to portions of the composite being bonded and portions of the composite having the distinct, tactile pattern that is unbonded, it is noted that the nonwoven composite described herein will also have a caliper delta value and a caliper ratio delta value. The bonded regions will possess a smaller caliper than the high loft regions. This value can be determined by selecting the particular (bonded or unbonded) region of the composite and measuring the caliper provided by the test procedure herein.

### Caliper Measurement Test

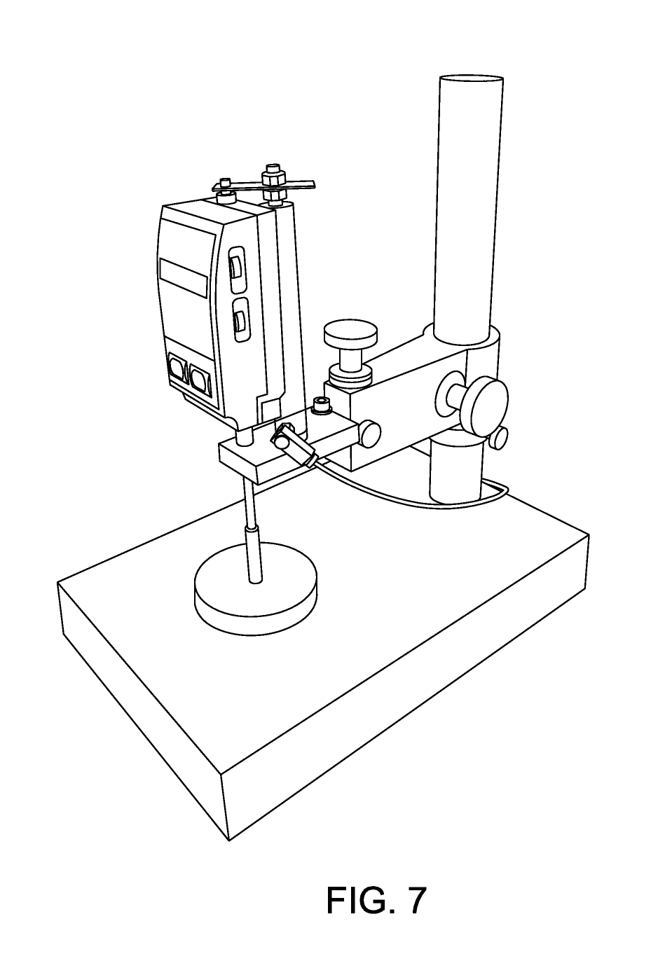

In order to test the caliper of a nonwoven material of the present invention, the following test was developed. Using a 76.2 mm diameter acrylic platen apparatus such as that shown in FIG. 7, the test is used to measure the thickness (or bulk) of a designated area of a nonwoven material or composite under a controlled loading pressure of about 0.345 kilopascal (kPa) (0.05 pound-force per square inch (psi)). The specified specimen size to be measured should be at least about 90 mm by about 102 mm. The data is recorded to the nearest 0.01 mm. The thickness of a nonwoven material is usually determined as the distance between an anvil or base and a circular platen used to apply the specified pressure. Thickness varies considerably depending on the pressure applied to the specimen when the thickness is measured; hence, it is essential to specify the pressure under which the thickness is measured.

When applicable, the lines on the caliper apparatus are in 5 mm increments and the millimeter distance is measured from the center of the circular platen. It is important that the correct standard tubing is used in order to assure that the tubing size does not affect the platen descent speed which should be about 3 seconds (+/−0.5 seconds). After 3 seconds, the value should be read and recorded. This procedure is used for each specimen needed for measurement. Again, the caliper ratio can be determined by taking the caliper of the fabric and dividing by the basis weight to give a value of mm/gsm.

Various articles may employ the embodiments of the present invention. For example, the nonwoven composite may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Absorbent articles may include a substantially liquid-impermeable layer (e.g., outer cover), a liquid-permeable layer (e.g., bodyside liner, surge layer, etc.), and an absorbent core. Besides liquid-permeable materials (e.g., liners, surge layers, etc.), the nonwoven composite of the present invention may have a wide variety of other uses, such as in providing an elastic waistband, leg cuff/gasketing (leg band), stretchable ear, side panel, outer cover, or any other component in which elastic properties are desirable. Various embodiments of an absorbent article, particularly a training pant that may be formed utilizing the nonwoven composite of the present invention are described in U.S. Pat. No. 8,343,127 to Dimitrijevs, et al. or U.S. Pat. No. 4,940,464 to Van Gompel et al.

Another embodiment of the present invention may be to employ the nonwoven composite as a bodyside liner which is generally used to help isolate the wearer's skin from liquids held in the absorbent core. For example, the liner presents a body facing surface that is typically compliant, soft feeling, and non-irritating to the wearer's skin. Typically, the liner is also less hydrophilic than the absorbent core so that its surface remains relatively dry to the wearer. The liner may be liquid-permeable to permit liquid to readily penetrate through its thickness. Exemplary liner constructions that contain a nonwoven web are described in U.S. Pat. No. 5,192,606 to Proxmire, et al.; U.S. Pat. No. 5,702,377 to Collier, IV, et al.; U.S. Pat. No. 5,931,823 to Stokes, et al.; U.S. Pat. No. 6,060,638 to Paul, et al.; and U.S. Pat. No. 6,150,002 to Varona, as well as U.S. Patent Application Publication Nos. 2004/0102750 to Jameson; 2005/0054255 to Morman, et al.; and 2005/0059941 to Baldwin, et al.

Another embodiment is as the outer cover of an absorbent article. The outer cover is typically formed from a material that is substantially impermeable to liquids. For example, the outer cover may be formed from a thin plastic film or other flexible liquid-impermeable material such as a polyethylene film. The film may be impermeable to liquids, but permeable to gases and water vapor (i.e., “breathable”). This permits vapors to escape from the absorbent core, but still prevents liquid exudates from passing through the outer cover. The nonwoven composite of the present invention may give a more cloth-like feeling that is desirable to many end-users.

An absorbent article utilizing the nonwoven composite of the present invention may also include a pair of side panels (or ears) that extend from the side edges of the absorbent article into one of the waist regions. The side panels may be integrally formed with a selected absorbent article component. For example, the side panels may be integrally formed with the outer cover or from the material employed to provide the top surface. In alternative configurations, the side panels may be provided by members connected and assembled to the outer cover, the top surface, between the outer cover and top surface, or in various other configurations. If desired, the side panels may be elasticized or otherwise rendered elastomeric by use of the elastic nonwoven composite of the present invention. Examples of absorbent articles that include elasticized side panels and selectively configured fastener tabs are described in PCT Patent Application WO 95/16425 to Roessler; U.S. Pat. No. 5,399,219 to Roessler et al.; U.S. Pat. No. 5,540,796 to Fries; and U.S. Pat. No. 5,595,618 to Fries.

As is typical, an absorbent article embodying the composite of the present invention may also include a pair of containment flaps that are configured to provide a barrier and to contain the lateral flow of body exudates. The containment flaps may be located along the laterally opposed side edges of the bodyside liner adjacent the side edges of the absorbent core. The containment flaps may extend longitudinally along the entire length of the absorbent core, or may only extend partially along the length of the absorbent core. When the containment flaps are shorter in length than the absorbent core, they may be selectively positioned anywhere along the side edges of diaper in a crotch region. In one embodiment, the containment flaps extend along the entire length of the absorbent core to better contain the body exudates. Such containment flaps are generally well known to those skilled in the art. For example, suitable constructions and arrangements for the containment flaps are described in U.S. Pat. No. 4,704,116 to Enloe.

To provide improved fit and to help reduce leakage of body exudates, the absorbent article may be elasticized with suitable elastic members, as further explained below. For example, the absorbent article may include leg elastics constructed to operably tension the side margins of the diaper to provide elasticized leg bands which can closely fit around the legs of the wearer to reduce leakage and provide improved comfort and appearance. Waist elastics may also be employed to elasticize the end margins of the absorbent article to provide elasticized waistbands. The waist elastics are configured to provide a resilient, comfortably close fit around the waist of the wearer. The elastic nonwoven composite of the present invention is suitable for use as the leg elastics and waist elastics. Exemplary of such materials are laminate sheets that either comprise or are adhered to the outer cover so that elastic constrictive forces are imparted thereto.

The various regions and/or components of the absorbent article may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the outer cover and bodyside liner are assembled to each other and to the absorbent core using an adhesive. Alternatively, the absorbent core may be connected to the outer cover using conventional fasteners, such as buttons, hook and loop type fasteners, adhesive tape fasteners, and so forth. Similarly, other absorbent article components, such as the leg elastic members, waist elastic members and sometimes even fasteners, may also be assembled into the absorbent article using any attachment mechanism. Although various configurations of an absorbent article have been described above, it should be understood that other absorbent article configurations are also included within the scope of the present invention. In addition, any absorbent article may be formed in accordance with the present invention, including, but not limited to, personal care absorbent articles, such as training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Several examples of such absorbent articles are described in U.S. Pat. No. 5,649,916 to DiPalma, et al.; U.S. Pat. No. 6,110,158 to Kielpikowski; U.S. Pat. No. 6,663,611 to Blaney, et al. Still other suitable articles are described in U.S. Patent Application Publication No. 2004/0060112 Al to Fell et al., as well as U.S. Pat. No. 4,886,512 to Damico et al.; U.S. Pat. No. 5,558,659 to Sherrod et al.; U.S. Pat. No. 6,888,044 to Fell et al.; and U.S. Pat. No. 6,511,465 to Freiburger et al. The present invention is particularly beneficial to absorbent articles having a front panel, two side panels, and a back panel. The distinct, tactile patterns can provide directional aid to an end user so that the user can identify and distinguish the front panel from the back panel for proper use of the absorbent article.

The advantages of the present invention are improved tactile properties that create better comfort to the end user. Again, the tactile properties can offer visual cues that may aid in the direction and/or operation of the article. For example, the unbonded areas can create a distinct, tactile pattern to distinguish the front of the article from the back to ensure proper use of the article. It may also offer direction to a user on where to best grasp the article to pull up and wear the article. Overall, the present invention is an improvement to existing bonded materials resulting in a nonwoven composite and article that is beneficial to the end consumer. As shown in FIGS. 5 and 6, the unbonded area provides a high, lofty pattern adjacent to an elastomeric polymer elastic film resulting in a functional, yet aesthetically pleasing composite. A variety of patterns can be used in the overall nonwoven composite. Such patterns may include, but are not limited to, flowers, squares, circles, stars, zig-zags, arrows, cartoon characters, faces, balloons, animals, nature, waves, swirls, rectangles, ovals, triangles, diamonds, polygons, and abstract shapes, caricatures, and the like. It is also important to note that the pattern of the present invention is not created through embossing techniques or by press-welded lines as described in as U.S. Patent Application Publication No. 2001/0008683 to Takai, et al. Rather, the present invention provides a novel way in which the voids, i.e. the unbonded areas of film and nonwoven web, provide the resulting distinct, tactile patterns.

### EXAMPLES

The following examples further describe and demonstrate various embodiments within the scope of the present invention. The examples are given solely for the purpose of illustration and are not to be construed as a limitation of the present invention, as many variations thereof are possible without departing from the spirit and scope of the invention.

### Example 1

The ability to form an elastic nonwoven composite was demonstrated. The elastic film was formed from 98 wt. % of VISTAMAXX™ 6102FL (ExxonMobil Chemical Co.) and 2 wt. % of SCC 79594 pigment concentrate (Standridge Color Corp.). VISTAMAXX™ 6102FL is a olefinic polypropylene elastomer composed of isotactic units with random ethylene distribution of about 16% having a density of 0.862 grams per cubic centimeter, a Vicat softening temperature of 126° F. (53° C.), and a melt index of 3.0 grams per 10 minutes (190° C., 2.16 kg). The SCC 79594 pigment contained titanium dioxide and other pigments to achieve a targeted tan color in a blended letdown with polypropylene resin (Exxon 3155).

After compounding, the polymer composition was extruded at a melt temperature of 403° F. and cast onto a chill roll (set to a temperature of 60° F.) operating at a speed of about 50 feet per minute. The film was then thermally bonded between two polypropylene spunbond facings having a basis weight of approximately about 15 grams per square meter. Specifically, the film and facings were fed between an anvil and patterned roll. The patterned roll was heated to a roll surface temperature of 250° F., the anvil roll was heated to a roll surface temperature of 250° F., and the pressure was 33 psi. The anvil and pattern rolls operated at a speed of 238 feet per minute so that the film was stretched in the machine direction at a stretch ratio of about 4.8 (i.e., 4.8 times its original length). Finally, the composite was transferred to a winder, which operated at a speed of 108 feet per minute to allow the composite to retract. The final basis weight was approximately 96 grams per square meter with 35 grams per square meter being comprised of the VISTAMAXX pigmented elastic film blend.

### Caliper Values

The caliper values were measured using a 76.2 mm diameter acrylic platen apparatus such as that shown in FIG. 7 under a controlled loading pressure of about 0.345 kilopascal (kPa) (0.05 pound-force per square inch (psi)). The values were read 5 times and the highest and lowest values were recorded for each example. The data was recorded to the nearest 0.01 mm.

### Example 2

FIG. 1 was made using the above exemplified process wherein two layers of spunbond having a basis weight of 13.5 gsm per layer was used to make a final composite having a basis weight of 80-85 gsm. Additionally, the calender rolls were set at a pressure of 30 psi, a temperature of 230° F. and a speed of 125 fpm. The caliper was found to be 2.7 mm-3.1 mm and the caliper ratio was found to be 0.032 mm/gsm to 0.036 mm/gsm.

### Example 3

FIG. 2 was made using the above exemplified process wherein two layers of spunbond having a basis weight of 12 gsm per layer was used to make a final composite having a basis weight of 115 gsm. Additionally, the calender rolls were set at a pressure of 30 psi, a temperature of 240° F. and a speed of 130 fpm. The caliper was found to be 4.8 mm-5.2 mm and the caliper ratio was found to be 0.047 mm/gsm to 0.050 mm/gsm.

### Example 4

### Comparative

FIG. 4 was made to compare the difference and lack of loft and patterns as shown in the examples of the present invention (Examples 1 and 2). Using the above exemplified process, the two layers of spunbond having a basis weight of 13.5 gsm per layer was used to make a final composite having a basis weight of 80-85 gsm. Additionally, the calender rolls were set at a pressure of 30 psi, a temperature of 230° F. and a speed of 125 fpm. The caliper was found to be 1.0 mm-1.1 mm and the caliper ratio was found to be 0.012 mm/gsm to 0.013 mm/gsm.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nonwoven composite comprising an elastomeric polymer elastic film positioned adjacent to and melt fused under heat and pressure to a nonwoven web material at a plurality of discrete bond points, wherein said nonwoven composite comprises a plurality of unbonded areas such that the unbonded areas provide unbonded elastic film positioned adjacent to but not fused to said nonwoven web such that the unbonded areas create distinct, tactile patterns in about 20% to about 75% of the surface area of the overall nonwoven composite and provides the nonwoven composite with a caliper ratio of from about 0.025mm/gsm to about 0.050 mm/gsm.

2. The nonwoven composite of claim 1 wherein said film comprises a plurality of apertures proximately adjacent to said bond points.

3. The nonwoven composite of claim 1 wherein the nonwoven web material comprises spunbond fibers, meltblown fibers, staple fibers, or combinations thereof.

4. The nonwoven composite of claim 1 wherein the unbonded areas create a distinct, tactile pattern in about 50% to about 75% of the surface area of the nonwoven composite.

5. The nonwoven composite of claim 1 having a basis weight of at least about 20 gsm.

6. The nonwoven composite of claim 1 wherein the elastic film is positioned between the nonwoven web material and an additional nonwoven web material.

7. An absorbent article comprising an outer cover, a bodyside liner joined to the outer cover, and an absorbent core positioned between the outer cover and the bodyside liner, wherein the absorbent article includes the nonwoven composite of claim 1.

8. The absorbent article of claim 7, wherein the outer cover, the liner and absorbent core form a chassis, at least a portion of the chassis comprises the nonwoven composite of claim 1.

9. The absorbent article of claim 7, wherein the outer cover includes the nonwoven composite of claim 1.

10. The absorbent article of claim 7, further comprising a waist band, leg band, or both that includes the nonwoven composite of claim 1.

11. The absorbent article of claim 7 wherein the distinct, tactile pattern in the overall nonwoven composite has a shape selected from flowers, squares, circles, stars, zig-zags, arrows, cartoon characters, faces, balloons, animals, nature, waves, swirls, rectangles, ovals, triangles, diamonds, polygons, and abstract shapes, caricatures, and combinations thereof.

12. The absorbent article of claim 10 further comprising a front panel, two side panels, and a back panel and wherein the distinct, tactile pattern directionally aids a user to distinguish the front panel from the back panel for proper use and operation of the absorbent article.
